(19) United States
(12) Patent Application Publication
(10) Pub. No.: US 2015/0335413 A1
(43) Pub. Date: Nov. 26, 2015

(54) MEDICAL INSTRUMENTS KIT AND METHOD FOR TREATING UTERUS PROLAPSE

(71) Applicant: Saba Nahedd, Ilfov (RO)

(72) Inventor: Saba Nahedd, Ilfov (RO)

(21) Appl. No.: 14/421,302

(22) PCT Filed: Jul. 25, 2013
(86) PCT No.: PCT/RO2013/000014

§ 371 Date: Feb. 12, 2015

(30) Foreign Application Priority Data
Oct. 31, 2012 (RO) A 2012 00777

(51) Int. Cl. A61F 2/00; A61B 17/3211; A61B 17/3201

(52) U.S. Cl. CPC A61F 2/0045; A61B 17/3201; A61B 17/3211

(57) ABSTRACT

The invention relates to a medical instruments kit for a surgical intervention with a view to treating the advanced anterior vaginal wall prolapse, very large cystocele, named “cystocele permagna” and the stage II and III uterus prolapse. The medical instruments kit comprises a clamp (A) for anchoring the uterine isthmus and some isthmic and suburethral strips (B and C), respectively, the clamp (A) consisting of two long rigid arms (1 and 2) provided with some posterior portions (a and b) spaced apart from each other, joined at the lower side by means of a bridge (3), in front of which the distance between the arms (1 and 2) has the maximum value, the latter being non-removably joined together to form an anterior portion (c), tapered towards an anterior smooth blunt tip (d), in the immediate proximity thereof, in a proximal curved zone (g) of the anterior portion (c) there being cut some upper and lower orifices (e and f), respectively, in front of the bridge (3) the long arms (1 and 2) being distally provided with two left side and right side rings (4 and 5), respectively, an additional ring (6) being integrally attached to a lower portion (i) of the right side ring (5), to the outer side of the long arms (1 and 2), in the proximity of the bridge (3) and before the beginning of the anterior portion (c) there being located two short left side and right side arms (7 and 8), respectively, said arms ending with some smooth blunt tips (g and h). The medical instruments kit is used for reconstructing the adjacent anatomical connections for the degree II or degree III uterus prolapse and for the advanced cystocele permagna, respectively.

The invention relates to a medical instruments kit for a surgical intervention with a view to treating the advanced anterior vaginal wall prolapse, very large cystocele, named “cystocele permagna” and the stage II and III uterus prolapse.

The uterus prolapse means the uterus descent into the vaginal axis and outside it, accompanied by the movement, to the same direction, of the vagina walls and of the adjacent portions of the urinary bladder and rectum. The descent is progressive into the small basin of the uterus, as a consequence of relaxation of the muscles and fibrous and extensible tissues of perineum—muscles forming the basin basis—as well as of the means of sustaining the organs of the small basin.

This explains the herniation, and the uterus prolapse, respectively, outside the vulvar commissure in various degrees, with the occurrence of the clinical symptoms consisting of heaviness in the lower portion of the pelvis, walking lumbar pain, urination and sexual intercourse pain, the sensation that “something is going to fall out/sits on a ball”, the introitus irritation and vagina ulcerations.

The uterus prolapse particularly affects the post-menopausal women, the women who had a difficult natural childbirth or several childbirths.

All these symptoms create a biological and social discomfort for the female patient.

The uterus prolapse is often associated to stress urinary incontinence, stress loss of some urine drops, sometimes masked by the excessive uterus prolabation.

It is worth mentioning that after any surgical treatment for correcting the uterus prolapse there must also be considered the correction of the stress urinary incontinence which is sometimes masked.

There are known medical instruments kits for a surgical intervention which consists in suspending the uterus at the promontorium—hysteropexy or sacropexy—while opening the peritoneal cavity, said kits containing a bistoury, some scissors, some Pean clamps, some Kocher clamps, a metallic bladder probe, some Farabeuf spacing devices, non-absorbable suturing threads, and a propylene mesh, some Pereyra-type clamps and needles or a Reverdyn-type clamp.

These kits have the disadvantages of not allowing to carry out, with full safety, an anatomical reconstruction surgical intervention without the occurrence of the pelvic pain syndrome, considered anatomically distorsional. Since the Pereyra clamps and needles penetrate from the upper edge of the pubic symphysis, crossing the aponeurosis of the rectus abdominis and retropubic muscles, reaching the vagina, there exists a great probability of injuring and perforating the urinary bladder the more so as they can not be oriented during the use.

With regard to the construction and use of the Reverdyn-type clamp, this is conceived so as to be inserted through the retropubic tunnels from the bottom to the top, oriented suprasynphysiarily to the aponeurosis of the rectus abdominis muscles, thus there existing a great probability of injuring and producing continuity solutions at the urinary bladder level, the more so as it can not be oriented during the use; this fact justifies the low surgical results, but always disputable, of the anatomical distorsion surgical intervention.

There are known methods for the treatment of the uterus prolapse, which comprise either a hysterectomy, which consists in extirpating the uterus through the vaginal route, or a hysteropexy, which consists in fixing the uterus to a stable element inside the small basin, by means of a thread or strips by fixing to a ligament of the first sacral vertebra, named promontofixation or by crossing the round ligaments which sustain the uterus, named ligamentary hysteropexy, either the anterior colporrhaphy and posterior colpoperineorrhaphy with the myorrhaphy of the annus raising muscles, which consists in reconstructing the muscles of the pelvic floor, in order to consolidate the pelvic organs support, or the triple Manchester operation consisting of the uterine cervix amputation, anterior colporrhaphy and posterior colpoperineorrhaphy.

The disadvantages of these methods consist in that either the vaginal vault prolapse occurs after some time, or they lead to the occurrence of a pelvic pain syndrome, having a relatively high percentage of recurrence, but none of the methods achieves the anatomical repositioning of the uterus into its normal position, so as to eliminate the unavoidable urinary incontinence when the urinary bladder structures have been affected by removing the uterus, and the uterus prolapse in most cases is solved partially, with relatively high chances of the prolapse recurrence after a while.

There are also known methods for treating the urinary bladder prolapse, named cystocele, which consist in inserting a permanent implant consisting of a central monofilament mesh made of polypropylene, placed between four arms of the same material.

The disadvantages of these methods consist in that they only deal with the cystocele, with a relatively low success, and result in the occurrence of a pain syndrome and do not solve the uterus prolapse.

The technical problem solved by the kit and the method according to the claimed inventions consists in safely performing the uterine isthmus anchoring to the rectus abdominis muscles sheath, within a reconstructive surgical intervention, for the restoration of the anatomical uterus connections and anatomical repositioning of the uterus into its normal position.

According to the invention, the kit solves the technical problem and eliminates the previously shown disadvantages in that it also comprises a clamp for passing the isthmus anchoring threads and some isthmic and suburetral strips, the clamp consisting of two long rigid arms provided with some posterior portions spaced apart from one another, united to the lower side, by means of a bridge, in front of which the distance between the arms has the maximum value, said arms being non-removably joined to each other to form an anterior portion, tapered towards an anterior smooth blunt tip in the immediate proximity thereof, in a proximal curved zone of the anterior portion there being cut some upper and lower orifices, respectively, in front of the bridge the long arms being provided distally with two left side and right side rings, respectively, to a lower portion of the right side ring there being integrally attached an additional ring, to the external side of the long arms, in the proximity of the bridge and before the beginning of the anterior portion, there being placed two short left side and right side arms, respectively, said arms ending with some smooth blunt tips.

The kit claimed by the invention also solves the technical problem in that the clamp for anchoring the uterine isthmus has a length of 28 cm, and from the level of the additional ring the long arms are rectilinear, for a length of 22 cm and curved for a length of 6 cm, with an angle of 30° in a proximal curved zone.

The kit claimed by the invention also solves the technical problem in that the short arms are located at a distance of 4 cm away from the bridge and have a length of 2.5 cm and make an angle alpha of 20° with the long arms.

The kit claimed by the invention also solves the technical problem in that at a distance of 1.0 cm away from the blunt tip of the proximal curved zone there are provided the upper and lower orifices, respectively, said orifices having a diameter of 3.0 . . . 4.0 mm, with a distance of 4.5 . . . 5.5 mm therebetween.

The kit claimed by the invention also solves the technical problem in that the isthmic strip is made of a medically acceptable non-absorbable material, such as propylene and consists of two long and short portions, respectively, the short portion being integrally connected to the long portion in front of a medial zone thereof, each of some ends of the long portion being linked with one of these threads made of the same non-absorbable material, and an inner end of the short portion being free.

The kit claimed by the invention also solves the technical problem in that the mesh size of the non-absorbable material that the long and short portions are made of is of 0.03 mm, the lengths of the portions are of 16.0 cm and 8.0 cm, respectively, and their width is of 1.2 cm.

The kit claimed by the invention also solves the technical problem in that the suburethral strip consists of a body made of polypropylene, each of some ends thereof being ligated with one of some threads made of polypropylene, the body length being of 10 cm and the width thereof being of 1.2 cm.

The method claimed by the invention solves the technical problem and eliminates the previously shown disadvantages in that the bladder-urethral junction is identified by the index finger of the right hand protected by a glove and at an angle of 45° made between the longitudinal side of the urethral meatus up to the posterior and horizontal vulvar commissure to penetrate paraurethrally, retropubicly and suprasymphyseally to carry out, in turn, one of the two right side and left side tunnels, respectively, up to the level of the rectus abdominis muscles sheath, then also continuing the incision circularly on the posterior face of the uterine cervix and removing the vaginal mucosa, followed by clamping, sectioning and ligating the cardinal ligaments, preferably of 1 cm and fixing the isthmus strip through the long portion, by suturing with the threads on the lateral and posterior faces of the uterine isthmus, and by means of the other threads the short, portion is sutured on the anterior face of the uterine isthmus, continuing with another suprapubic incision of a length of 4.0 . . . 5.5 cm, made by the bistoury, up to the proximity of the rectus abdominis muscles sheath, and then passing the threads, in turn, and the other threads, respectively, through one of the tunnels, penetrating into the rectus abdominis muscles sheath, said muscles being introduced, in turn, through the lower and upper orifices anteriorly provided in the proximal curved zone of the clamp, followed by performing the anterior colpectomy and anterior colporrhaphy as well as suturing the posterior incision on the uterine cervix, the thread ends of the body of the suburethral strip which is mounted at the cysto-urethral junction level are anchored to the rectus abdominis muscles sheath, to the external extremities of the suprapubic incision under the control of the graded urethral probe to obtain a urethra elongation by 1.5 cm medially, as against the anchoring of the ends of the suburethral strip threads, the ends of the isthmic strip threads being anchored to the rectus abdominis muscles sheath, thereafter pulling and ligating the ends of the isthmic strip threads up to bringing the utherine cervix back to its intermediary anatomical position as close as possible to the normal position, in the conditions in which by means of the short portion of the isthmic strip, the uterine isthmus is covered and fixed on the anterior face, and the end of the long portion is cut according to the patient's dimensions, then the thread is ligated and finally there is sutured the suprapubic incision and the posterior colpoperineorrhaphy with the myorrhaphy of the annus raising muscles.

The technical problem is also solved in that the medical instruments kit is used for reconstructing the adjacent anatomical connections for the degree II or degree III uterus prolapse, and for advanced cystocele permagna, respectively.

By applying the kit and method claimed by the inventions in the group of inventions the following advantages are obtained:
the clamp is easy to be handled and allows the threads of both strips to pass at the same time through a tunnel;
the use of non-absorbable polypropylene material for making the isthmic and suburethral strips and of the threads to be fixed to the rectus abdominis muscles sheath—a hormonally independent tissue—makes the recurrence risks as low as possible or almost inexistent;
there is solved the uterus prolapse and cystocele permagna and the uterus is brought back to its anatomical position;
placing the uterus in the intermediary normal position avoids the extension of the retrovaginal space and consequently prevents the occurrence of retrocele and elitrocele;
there is solved the stress urinary incontinence;
the surgical approach is exclusively on the vaginal route, thereby the incidents and accidents of opening the peritoneal cavity being avoided.

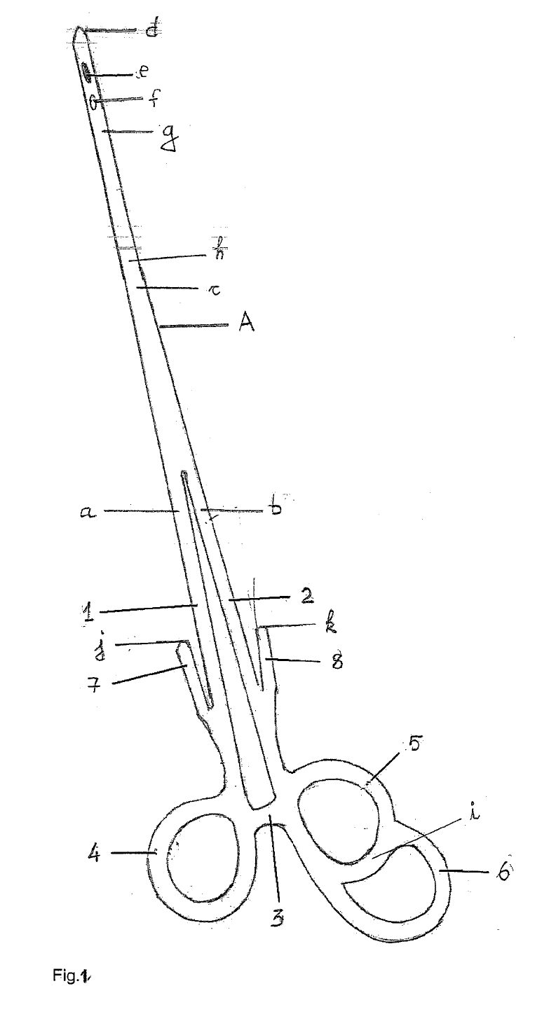

There are given hereinafter an embodiment for carrying out the kit and an embodiment for the method, respectively, as claimed by the inventions, in connection with FIG. 1 . . . 14, which represent:

FIG. 1 front view of a clamp for anchoring the uterine isthmus;

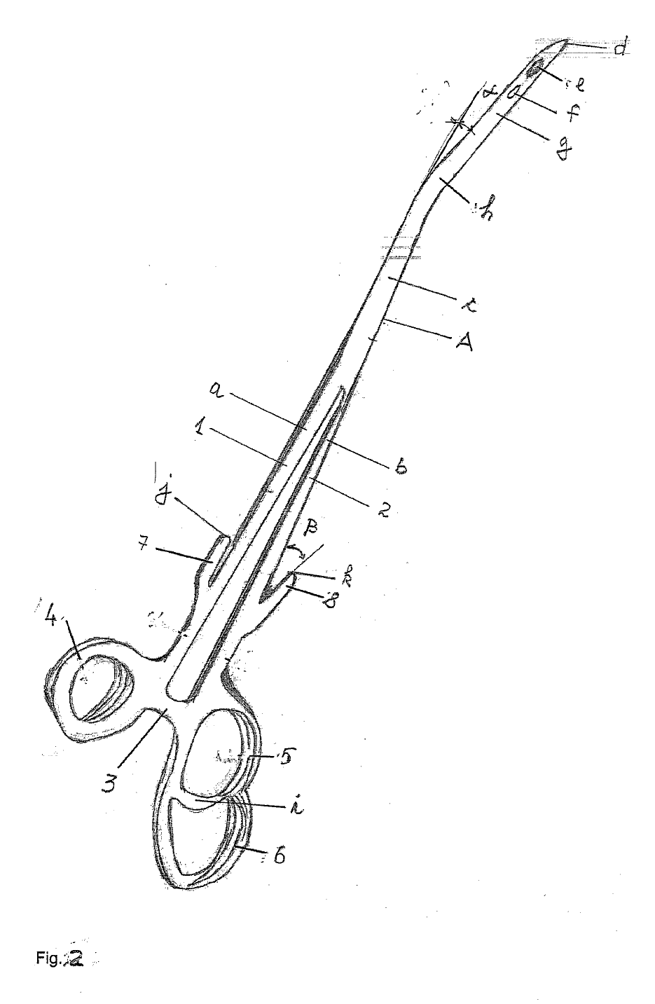

FIG. 2 lateral perspective view of the clamp for anchoring the uterine isthmus;

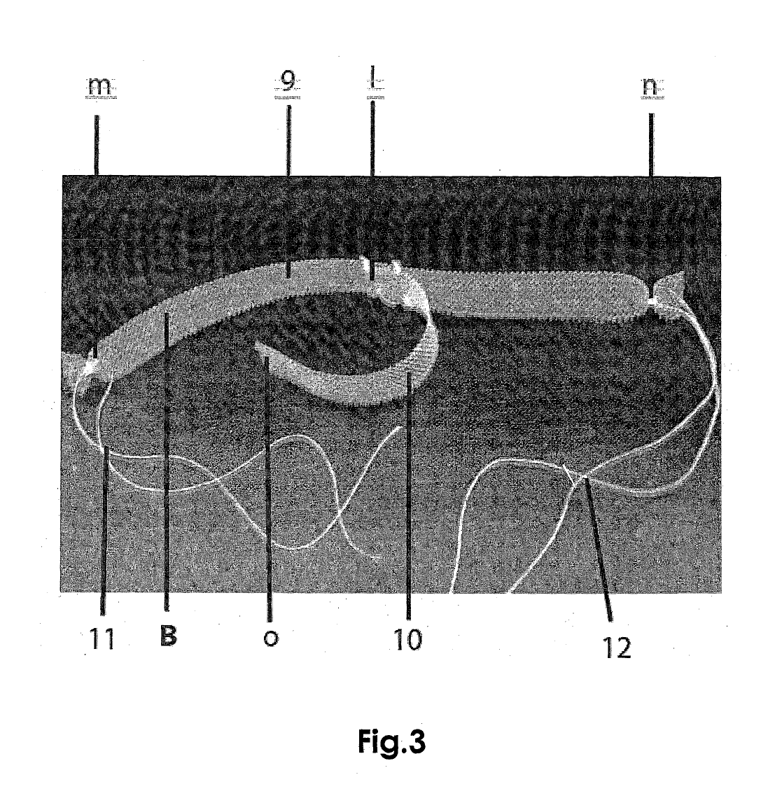

FIG. 3 lateral perspective view of an isthmus strip;

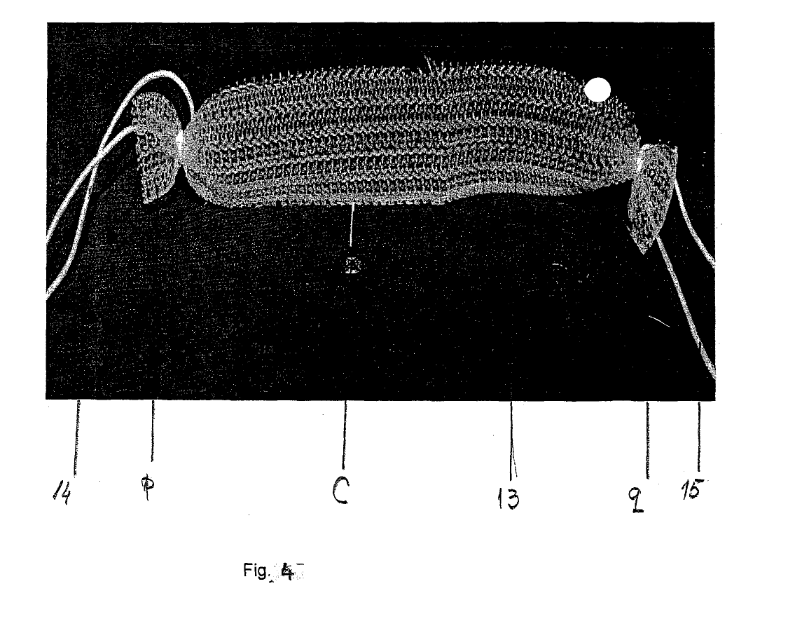

FIG. 4 lateral perspective view of a suburethral strip;

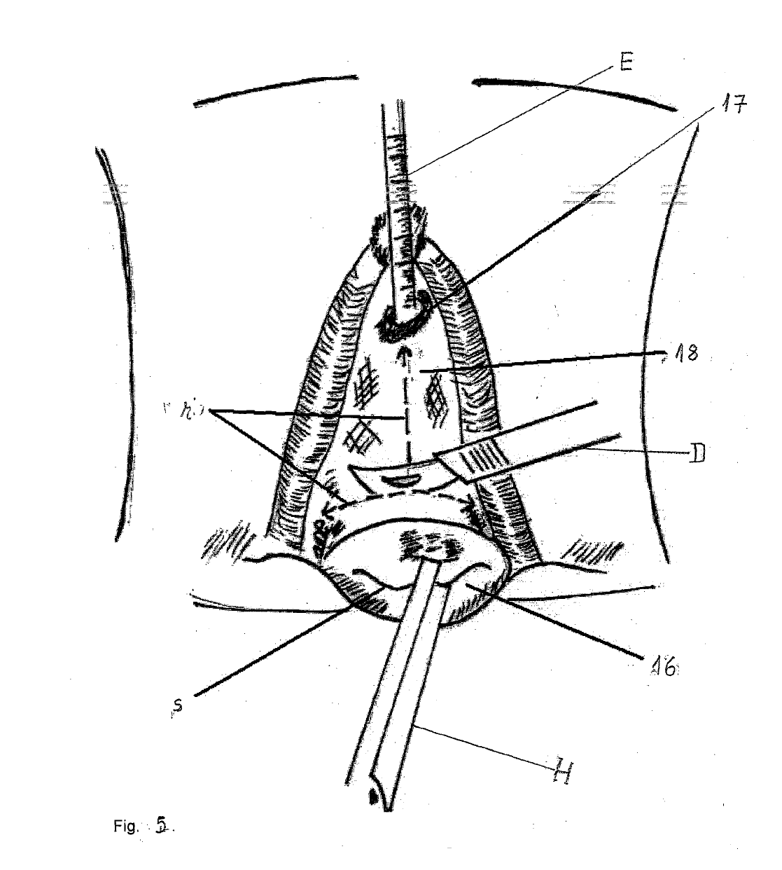

FIG. 5 schematic perspective view of carrying out, in time 1, a reversed T-shaped incision extending in the proximity of the external uterine orifice up to the urethral tubercle;

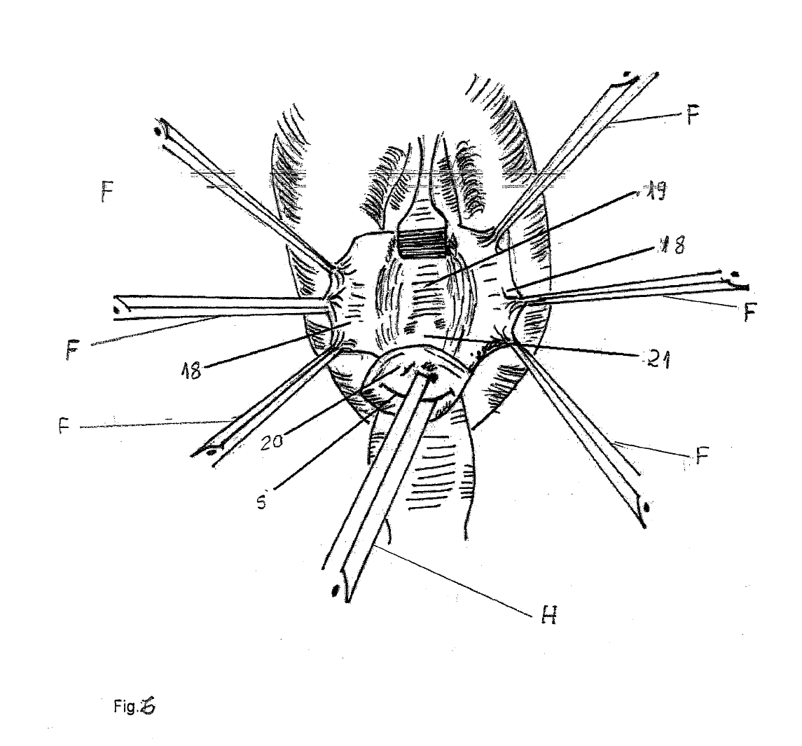

FIG. 6 a schematic view of carrying out, in time 2, the removal of the bladder from the anterior vaginal wall and of the bladder from the uterine cervix;

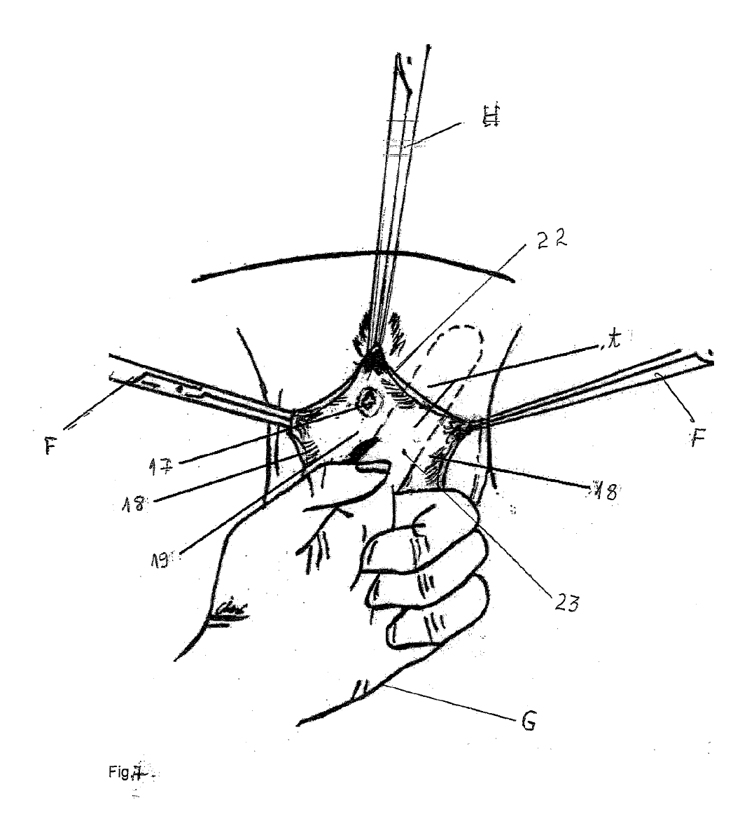

FIG. 7 schematic view of carrying out, in time 3, some retropubic tunnels;

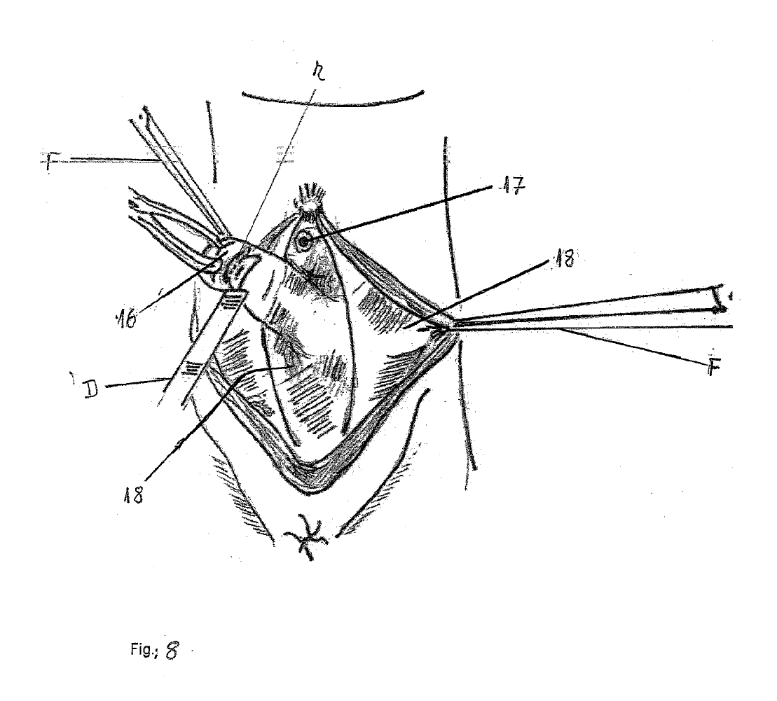

FIG. 8 schematic view of continuing, in time 4, the incision on the posterior face of the uterine cervix;

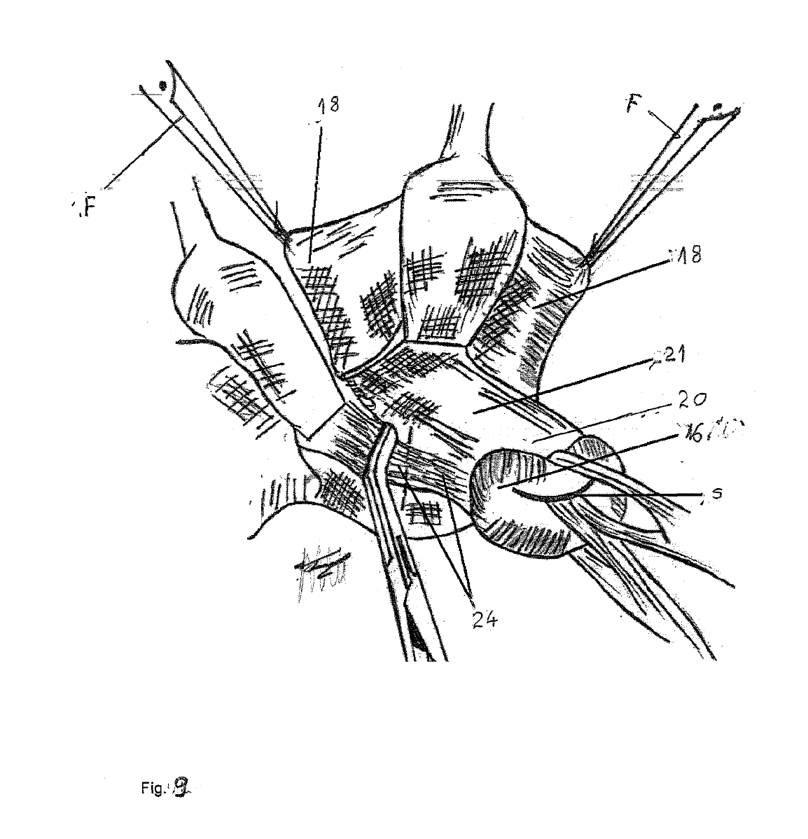

FIG. 9 schematic view of carrying out, in time 5, clamping, sectioning and ligating the cardinal ligaments;

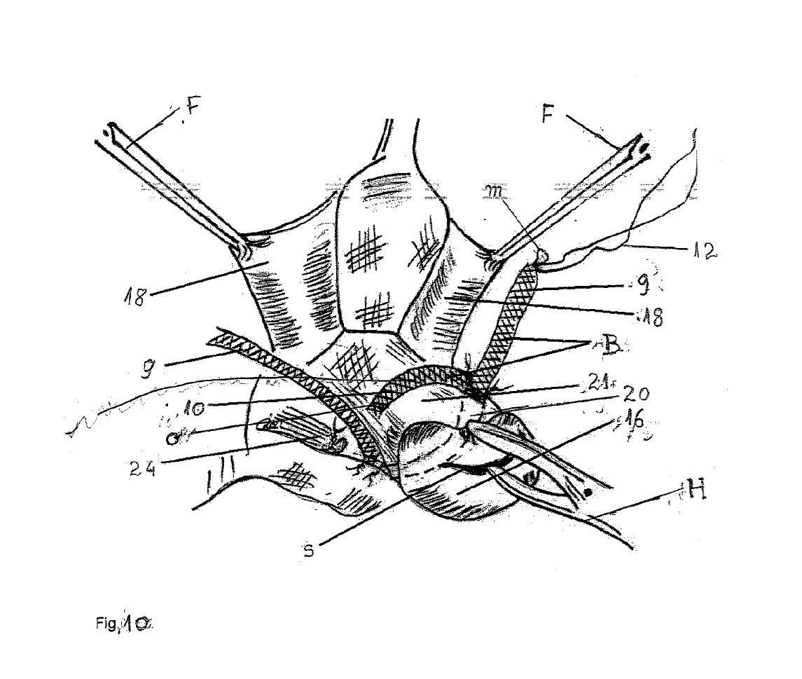

FIG. 10 schematic view of fixing, in time 6, a long isthmic strip onto the lateral and posterior faces of the uterine isthmus;

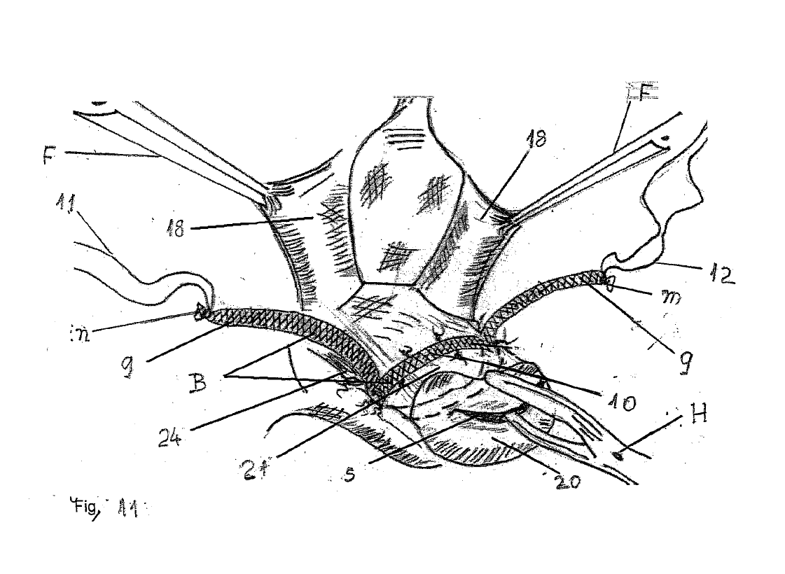

FIG. 11 schematic view of fixing, in time 7, the free short portion of the isthmic strip on the anterior face of the uterine isthmus;

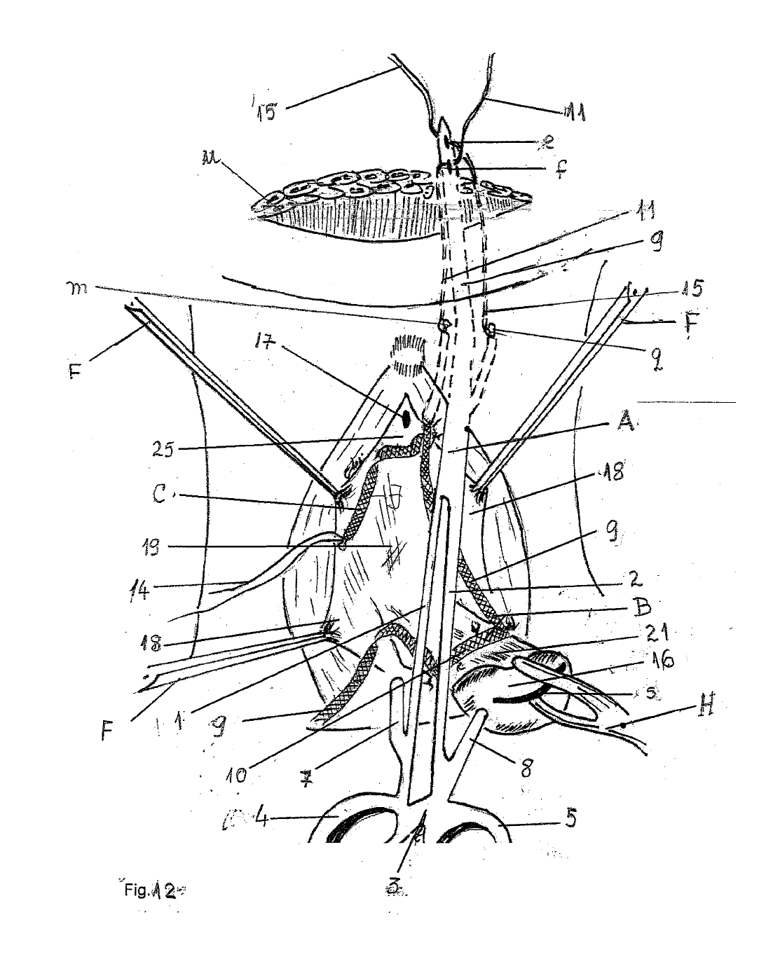

FIG. 12 schematic view of mounting, in time 8, by means of the clamp, the ends of the two threads of some suburethral strips and some isthmic strips, respectively, through the two orifices of the clamp, and of passing the same through the tunnels;

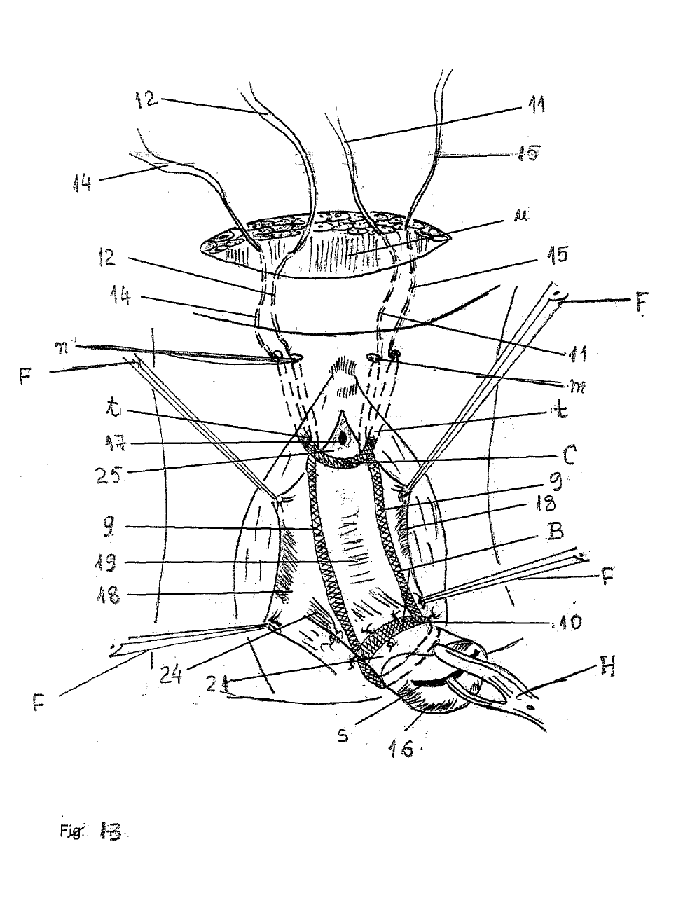

FIG. 13 schematic view of passing, in time 9, the ends of the suburethral strip to the external extremities of the suprapubic incision, and of the isthmic strip thread ends in the medial zone of the suprapubic incision;

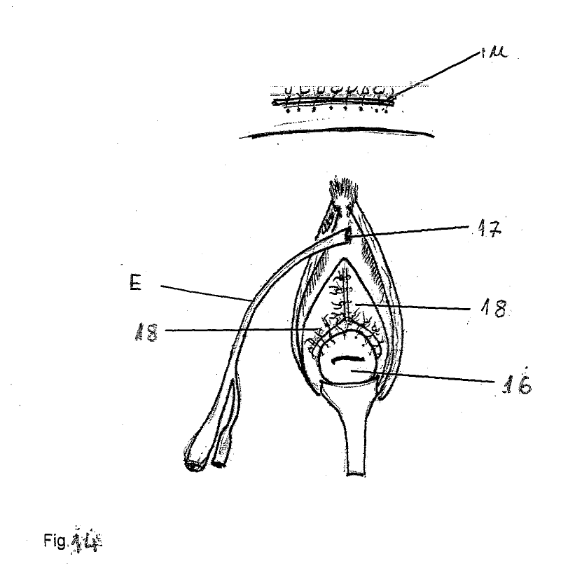

FIG. 14 schematic view of carrying out, in time 10, the suprapubic transverse incision suturing which represents the final image of the surgical intervention.

The kit claimed by the invention comprises a box not represented in the figures, which is provided with some compartments for accommodating a bistoury, 6 Pean clamps, 3 Kocher clamps, two surgical scissors, absorbable and non-absorbable suturing threads, a clamp A for anchoring the uterine isthmus, an isthmic strip B, a strip C for urethrocystopexy, a metallic bladder probe and two Farabeuf spacing devices, of which only the clamp A and strips B and C are represented in the figures, the other medical instruments and the strip C being known per se.

The clamp A consists of two long rigid arms 1 and 2 provided with some posterior portions a and b spaced apart from each other, joined at the lower side by means of a bridge 3, in front of which, between the arms 1 and 2 there is a distance which is reduced gradually until these make one piece.

The arms 1 and 2 are joined to each other by welding, forming an anterior portion c tapered towards an anterior smooth blunt tip d, the length of which is of 2.5 cm.

At a distance of 10 cm away from the tip d, in the portion c there is provided an upper orifice e with a diameter of 3.0 . . . 4.0 mm, and at a distance of 4.5 . . . 5.5 mm away from the centre of the orifice c there is provided a lower orifice f having the same diameter as the orifice e.

The portion c has a proximal curved zone g whose axis is inclined by an angle α of 30° in relation to the axis of a lower zone h.

The arms 1 and 2 are distally provided with two profiled left side and right side rings 4 and 5, located in front of the bridge 3. To a lower portion i of the ring 5 there is rigidly attached an additional ring 6.

To the outer side of the arms 1 and 2, at a distance of 4 cm away from the bridge 3, there are placed two short left side and right side arms 7 and 8, respectively, which make an angle β of 20° with the arms 1 and 2, each of them having a length of 25 cm. each of the short arms 7 and 8 ending with one of some smooth blunt tips j and k.

The total length of the clamp A is preferably of 28 cm, and from the level of the additional ring 6, the arms 1 and 2 are rectilinear on a length of 22 cm, then continuing with a proximal inclined zone g having a length of 6 cm.

The strip B is made up of a medically acceptable non-absobable material which can be polypropylene and which has a long portion 9, to which, in front of a medial zone I thereof, there is fixed a short portion 10. The size of the non-absorbable material meshes is preferably of 0.03 mm, the lengths of the portions 9 and 10 are equal to 16.0 cm and 8.0 cm, respectively, and the width of the portions 9 and 10 is equal to 1.2 cm. Each of some ends m and n of the portion 9 are ligated with one of some non-absorbable threads 11 and 12, and an interior end o of the portion 10 is free. The free end o of the portion 10 is cut during the surgical intervention, according to the patient's dimensions and it is then sutured below a long portion 9. The free portion 10 starts from a distance of 5.0 cm from the end m of the long portion 9.

The suburethral strip C consists of a body 13 made of the same medically acceptable non-absorbable material that the strip B is made of and has some ends p and q, each being ligated with one of some further non-absorbable threads 14 and 15. The length of the body 13 is of 10 cm and the width thereof is of 1.2 cm.

In order to anchor the uterine isthmus to the rectus abdominis muscles sheath as close as possible to the normal anatomical location, during the operating time I of a surgical operation, a reversed “T”-shaped incision r is made by means of the bistoury D starting from the level of the uterine cervix 16.

The incision r is carried out transversally with a length of 2.0 cm, 1.5 cm above an external uterine orifice s and then is continued along a direction perpendicular to the one along which it was previously carried out, so that finally the incision is reversed “T”-shaped up to the urethral meatus 17, wherein the metallic bladder probe E is inserted.

The incision r is carried out in the conditions in which a Pean clamp F is fixed at the level of the uterine cervix 16.

During the operating time II, by means of five Pean clamps F there takes place the removal, from the lateral side, of a vaginal mucosa 18 from the bladder 19 as well as of the latter from the anterior face of the uterine cervix 16 up to the level of an uterine isthmus 21.

During the operating time III, the bladder-urethral junction 22 is identified by means of the index finger 23 of the right hand G, protected by a glove and at an angle of 45° formed by the longitudinal side on the urethral meatus 17 up to the posterior and horizontal vulvar commissure 24, there takes place a paraurethral, retropubic and suprasymphyary penetration to carry out, in turn, one of the two right side and left side tunnels t, respectively. The right side tunnel t is made up to the level of a rectus abdominis muscles sheath. Then it is proceeded similarly for carrying out the left side tunnel t. During the operating time IV, by means of the bistoury D the incision r is also continued circularly on the posterior face of the uterine cervix 16, then using the index finger 23 of the right hand G, the vaginal mucosa 18 is removed from the rectum.

During the operating time V there are performed the clamping, sectioning and ligating the cardical ligaments 24, preferably of 1 cm.

During the opening time VI, the isthmic strip B having the long portion 9 is fixed by suturing with the non-absorbable threads 12 on the lateral and posterior faces of the uterine isthmus 21.

During the operating time VII, the short portion 10 of the isthmic strip B is sutured on the anterior face of the uterine isthmus 21 by means of the non-absorbable threads.

During the operating time VIII, there is performed, by means of the bistoury D a suprapubic incision u with a length of 4.0 . . . 5.5 cm, up to the proximity of the rectus abdominis muscles sheath.

During the operating time IX, there is used the clamp A for passing the threads 11 and 15 and 12 and 14 for anchoring the uterine isthmus, in turn, through the orifices f and e through the tunnels t.

During the operating time X, the end zone g of the clamp A is introduced through the right side tunnel t, penetrating through the rectus abdominis muscles sheath, then the zone g is introduced through the left side channel t penetrating through the rectus abdominis muscles sheath.

During the operating time XI, there are carried out the anterior colpectomy and then the anterior colporrhaphy which consist in resecting, by means of the bistoury D, the vaginal mucosa 18 in excess on either side and in the anterior vaginal suture, followed by suturing the posterior incision suture on the uterine cervix 16.

During the operating time XII, the ends of the non-absorbable threads 14 and 15 of the suburethral strip C which is mounted at the level of the cystourethral junction are anchored to the rectus abdominis muscles sheath, at the external extremities of the suprapubic incision u. These ends of the threads 14 and 15 are tied under the control of the graduated urethral probe E there being obtained al elongation of 1.5 cm of the urethra 25.

During the operating time XIII, there are anchored to the rectus abdominis muscles sheath the ends of the threads 11 and 12 of the medial isthmic strip B as against the anchoring of the threads 14 and 15 of the suburethral strip C.

During the operating time XIV, there takes place the pulling and ligating the ends of the threads 11 and 12 of the isthmic strip B up to bringing the uterine cervix 16 in its intermediary anatomical position as close as possible to the normal position, without pulling it too high, so as not to open the urethro-rectal space in order to avoid elitrorectocele.

By means of the portion 10 the uterine isthmus 21 is covered and fixed on the anterior face in order to prevent slipping or tilting the same, and the end o of the portion 10 is cut according to the patient's dimensions and is sutured under the portion 9.

During the operating time XV, the suprapubic transverse incision u suture takes place.

During the operating time XVI, the posterior colpoperineorrhaphy with the myorrhaphy of the annus raising muscles takes place.

The method claimed by the invention may also be applied on an uterine cervix 16 with benign lesions or hypertrophic elongation of the uterine cervic 16, and after fixing the isthmic strip B there takes place the amputation of the uterine cervix 16 by following the previously described operating times I . . . XVI.

The isthmic strip B which is made of a non-absorbable material constitutes a permanent implant, like a hammock having the uterine cervix 16 located thereon and which is fixed to the rectus abdominis muscles sheath by the ends of the threads 11 and 12, which make the recurrence not possible.

The clamp A is rigid, allowing to guide the blunt tip d which, in its turn, is smooth in order to avoid the vascular and visceral lesions, particularly those of the urinary bladder. The curvature of zone g is so chosen as to be adapted to be introduced and extracted into/from the retropubic tunnels t and to allow keeping direct contact with the posterior face of the pubic symphysis and externalizing the blunt tip d above the upper edge of the pubic symplysis.

The short arms 7 and 8 on the left and right side, respectively, are positioned parallelly to the urethral meatus 17 and allow the clamp A orientation both towards the right side and towards the left side, as against the urethral meatus 17, indicating the inclination angle of the clamp A corresponding to the angle of 45° of each of the retropubic tunnels t.

The additional ring 6 maintains the clamp A orientation, facilitating the perforation of the aponeurosis of the rectus abdominis muscles.

The presence of the orifices e and f in the zone g makes possible to pass the threads 11 and 15 and the threads 12 and 14 of the strips B and C, respectively.

By mounting the suburethral strip C at the level of the cystourethral junction the stress urinary incontinence is solved.

From the date of 25 Oct. 2012 up to the date of 30 Jun. 2013 there were hospitalized 7 cases in the Clinical Hospital “Polizu”, said cases after clinical and paraclinical investigations—“mictional cystography”—fractional curettage biopsy, resection with loop diathermy of the uterine cervix in order to exclude the associated pathology, there was diagnosed the uterus prolapse degree II-III, for which there was performed the uterine isthmus anchoring through the strip to the rectus abdominis muscles sheath, as claimed by the invention.

The first patient, 59 years of age, came to the hospital for stress urine loss, heaviness in the lower portion of pelvis, introitus irritation and ulceration.

After performing the clinical and paraclinical investigations, there was diagnosed genital prolapse degree II, stress urinary incontinence, and lesion cervix.

The uterine isthmus was anchored to the rectus abdominis muscles sheath by means of strips B and C.

A peculiarity of this case is the presence of a uterine cervix lesion requiring the uterine cervix amputation.

Post-surgery, the patient resumed the normal physiological micturition 3 days after the removal of the endovesical probe, subsequently having the bladder residual of about 30 ml, i.e. within the normal values. The patient was externalized 6 days post-surgery with healed condition.

Eight months after the surgery the patient came for examination with good bladder retention, upon the genital examination with valves, the uterine cervix was in its anatomical position, upon manoevering the valves the patient does not lose urine.

In other 5 case it was proceeded as claimed by the invention, with the uterine cervix amputation due to the lesions present thereon.

The seventh patient, 50 years of age, has as anatomo-clinical peculiarities a cystocele permagna, prolapse degree II with the following anatomo-pathological post fractional curettage biopsy and endocevical polyp resection result: “Endocervical fragments with polypoid aspect, endometrium fragments with simple endometrial hyperplasia”.

On the date of 20 Feb. 2013 it was proceeded as claimed by the invention with regard to anchoring, with the strip, the uterine isthmus to the rectus abdominis muscles sheath and urethrocystopexy.

In this case the uterine cervix amputation was not necessary, since the uterine cervix appears without pathological modifications demonstrated by Babe [symbol] -Papanicolau colposcopy and cytopathology, but having an endometrial polyp for which a month before the polyp resection operation and fractional curettage biopsy were performed, the anatomo-pathological results showing the benignity thereof.

In all the previously mentioned cases there was performed the pre-surgery and post-surgery mictional cystography. The post-surgery mictional cystography showed the lower pole of the urinary bladder with irregular contour which is situated at the level of pubic symphysis, i.e. the ascent of this pole that was much previously lowered below the lower margin of pubic symphysis.

All the patients were externalized 6-7 days post-surgery with good bladder retention and bladder residual in the range of 0-50 ml and the disappearance of all symptoms accused upon hospitalization, such as: stress urine loss, disappearance of heaviness in the lower portion of pelvis, the sensation that “something falls out/sits on the ball”, healing of intruitus irritation and vagina ulceration, disappearance of lumbar walking and urination pains.

In the patients that returned for examination after 4-5 months, the clinical examination by the use of the valves showed that the uterine cervix is situated in its normal position, while keeping the retention during the valve manoeuvering and the disappearance of all symptoms they accused before the surgery.

These patient will be monitored for a period of minimum 1-3 years in order to demonstrate that the recurrence after the intervention as claimed by the invention does not exist, since the technique is based on a polypropylene material and non-absorbable threads, anchored to the rectus abdominis muscles sheath, which is a hormonally independent tissue which does not relax by aging.

1. Medical instruments kit comprising a bistoury, two surgical scissors, six Pean clamps, three Kocher clamps, a metallic bladder probe, two Farabeuf spacing devices, characterized in that it also comprises a clamp (A) for anchoring the uterine isthmus and some isthmic and suburethral strips (8 and C), respectively the clamp (A) consisting of two long rigid arms (1 and 2) provided with some posterior portions (a and b) spaced apart from each other, joined at the lower side by means of a bridge (3), in front of which the distance between the arms (1 and 2) has the maximum value, the latter being non-removably joined together to form an anterior portion (c), tapered towards an anterior smooth blunt tip (d), in the immediate proximity thereof, in a proximal curved zone (g) of the anterior portion (c) there being cut some upper and lower orifices (e and f), respectively, in front of the bridge (3) the long arms (1 and 2) being distally provided with two left side and right side rings (4 and 5), respectively, an additional ring (6) being integrally attached to a lower portion (i) of the right side ring (5), to the outer side of the lone, arms (1 and 2), in the proximity of the bridge (3) and before the beginning of the anterior portion (c) there being located two short left side and right side arms (7 and 8), respectively, said arms ending with some smooth blunt tips (g and h).

2. Kit according to claim 1, characterized in that the clamp (A) for anchoring the uterine isthmus has a length of 28 cm, and at the level of the additional ring (6) the long arms (1 and 2) are rectilinear for a length of 22 cm and curved on a length of 6 cm, with an angle of 30° in a proximal curved zone (g).

3. Kit according to claim 1, characterized in that the short arms (7 and 8) are located at a distance of 4 cm from the bridge (3) and have a length of 2.5 cm and form an angle β of 20° with the long arms (1 and 2).

4. Kit according to claim 1, characterized in that at a distance of 1.0 cm from the blunt tip (d) of the proximal curved zone (g) there are provided the anterior and posterior orifices (e and f), respectively, said orifices having a diameter of 4.5 . . . 5.5 mm.

5. Kit according to claim 1, characterized in that the isthmic strip (B) is made of a medically acceptable non-absorbable material, such as propylene and consists of two long and short portions (9 and 10), respectively, the short one being integrally attached to the long portion (9) in front of a medial zone (I) thereof, some ends (m and n) of the long portion (9) being linked by one of some threads (11 and 12) made of the same non-absorbable material, and an inner end (o) of the short portion (10) being free.

6. Kit according to claim 5, characterized in that the size of the meshes of the non-absorbable material that the long and short portions (9 and 10) are made up of is of 0.03 mm, the lengths of the portions (9 and 10) are of 16.0 cm and 8.0 cm, respectively, and their width is of 1.2 cm.

7. Kit according to claim 1, characterized in that the suburethral strip (C) consists of a body (13) made of polypropylene which has some ends (p and q), each tied with one of some threads (14 and 15) made of polypropylene the length of body (13) being of 10.0 cmand the width thereof being of 1.3 cm.

8. Method for the treatment of uterus prolapse, as claimed by the invention, carried out by means of the kit as claimed in claim 1, which comprises carrying out the uterine isthmus anchoring as close as possible to the normal anatomical location, consisting in making, by means of a bistoury, a reversed “T”-shaped incision of the anterior portion of the uterine cervix, followed by removing, from the lateral side, the vaginal mucosa from the bladder, and the latter from the uterine cervix up to the uterine isthmus level, characterized in that further on the bladder-urethral junction (22) is identified by means of the index finger (23) of the right hand (G) protected by a glove, and at an angle of 45° formed by the longitudinal side from the urethral meatus (17) up to the posterior and horizontal vulvar commissure (24) there is penetrated in a paraurethral, retropubic and suprasymplysiarily in order to carry out one of the two right side and left side tunnels (t), respectively, up to the level of the rectus abdominis muscles sheath, followed by also continuing the incision (r) circularly on the posterior face of the uterine cervix (16) and removing the vaginal mucosa (18), followed by clamping, sectioning and ligating the cardinal ligaments (24) of preferably 1 cm and fixing the isthmic strip (6) through the long portion (9) by suturing with the threads (12) from the lateral and posterior sides of the uterine isthmus (21), and through the other threads (11), the short portion (10) is sutured on the anterior face of the uterine isthmus (21), followed by making, by means of the bistoury (D), another suprapubic incision (u) with a length of 4.0 . . . 5.5 cm up to the proximity of the restus abdominis muscles and then, the threads (11 and 15) and the other threads (12 and 14), respectively, each are passed. in turn, through one of the tunnels (t), penetrating into the rectus abdominis muscles sheath, which are introduced in turn, anteriorly through the upper and lower orifices (f and e), respectively, said orifices being provided in the proximal curved zone (g) of the clamp (A), followed by carrying out the anterior colpoctomy and the anterior colporrhaphy, as well as the suturing of the posterior incision on the uterine cervix (16), then the ends of threads (14 and 15) of the body (13) of the suburethral strip (C), which is mounted at the level of the cysto-urethral junction, are anchored to the rectus abdominis muscles sheath at the external extremities of the suprapubic incision (u), under the control of the graded urethral probe (E) there being obtained an elongation of the urethra (25) by 1.5 cm, medially, as against the anchoring of the ends of the threads (14 and 15) of the suburethral strip (C), the ends of threads (11 and 12) of the isthmic strip (B) being anchored to the rectus abdominis muscles sheath (C), followed by pulling and ligating the ends of threads (11 and 12) of the isthmic strip (13) until the uterine cervix (16) is brought to its intermediary anatomical position as close as possible to the normal position, in the conditions in which, by means of the short portion (10) of the isthmic strip (13) the uterine isthmus (21) is covered and fixed on the anterior face, and the end (o) of the short portion (10) being cut according to the patient's dimensions, then it is fixed under the long portion (9) in the uterine isthmus (21) and finally suturing the suprapubic incision (r) and the posterior colpoperineorrhaphy with the myorrhaphy of the annus raising muscles.

9. Medical instruments kit according to claim 1, which is used for reconstructing the adjacent anatomical connections for a degree II or degree III uterus prolapse, and for an advanced cystocele permagna, respectively.

10. Kit according to claim 2 characterized in that at a distance of 1.0 cm from the blunt tip (d) of the proximal curved zone (g) there are provided the anterior and posterior orifices (e and f), respectively, said orifices having a diameter of 4.5 . . . 5.5 mm.

11. Medical instruments kit according to claim 2, which is used for reconstructing the adjacent anatomical connections for a degree II or degree III uterus prolapse, and for an advanced cystocele permagna, respectively.

12. Medical instruments kit according to claim 3, which is used for reconstructing the adjacent anatomical connections for a degree II or degree III uterus prolapse, and for an advanced cystocele permagna, respectively.

13. Medical instruments kit according to claim 4, which is used for reconstructing the adjacent anatomical connections for a degree II or degree III uterus prolapse, and for an advanced cystocele permagna, respectively.

14. Medical instruments kit according to claim 5, which is used for reconstructing the adjacent anatomical connections for the a degree II or degree III uterus prolapse, and for the an advanced cystocele permagna, respectively.

15. Medical instruments kit according to claim 6, which is used for reconstructing the adjacent anatomical connections for a degree II or degree III uterus prolapse, and for an advanced cystocele permagna, respectively.

16. Medical instruments kit according to claim 7, which is used for reconstructing the adjacent anatomical connections for a degree II or degree III uterus prolapse, and for an advanced cystocele permagna, respectively.
